SEQUENCE LISTING (35 sequences)

SEQ ID NO: 1 - DNA, 405 bp, Aspergillus nidulans
  CDS (1)..(405): basf-dewA hydrophobin
atgcgcttcatcgtctctctcctcgccttcactgccgcggccaccgcgaccgccctcccggcctctgccgcaaagaacgcgaagctggccacctcggcggccttcgccaagcaggctgaaggcaccacctgcaatgtcggctcgatcgcttgctgcaactcccccgctgagaccaacaacgacagtctgttgagcggtctgctcggtgctggccttctcaacgggctctcgggcaacactggcagcgcctgcgccaaggcgagcttgattgaccagctgggtctgctcgctctcgtcgaccacactgaggaaggccccgtctgcaagaacatcgtcgcttgctgccctgagggaaccaccaactgtgttgccgtcgacaacgctggcgctggtaccaaggctgag

SEQ ID NO: 2 - protein, 135 aa, Aspergillus nidulans
  basf-dewA hydrophobin
MRFIVSLLAFTAAATATALPASAAKNAKLATSAAFAKQAEGTTCNVGSIACCNSPAETNNDSLLSGLLGAGLLNGLSGNTGSACAKASLIDQLGLLALVDHTEEGPVCKNIVACCPEGTTNCVAVDNAGAGTKAE

SEQ ID NO: 3 - DNA, 471 bp, Aspergillus nidulans
  CDS (1)..(471): basf-rodA hydrophobin
atgaagttctccattgctgccgctgtcgttgctttcgccgcctccgtcgcggccctccctcctgcccatgattcccagttcgctggcaatggtgttggcaacaagggcaacagcaacgtcaagttccctgtccccgaaaacgtgaccgtcaagcaggcctccgacaagtgcggtgaccaggcccagctctcttgctgcaacaaggccacgtacgccggtgacaccacaaccgttgatgagggtcttctgtctggtgccctcagcggcctcatcggcgccgggtctggtgccgaaggtcttggtctcttcgatcagtgctccaagcttgatgttgctgtcctcattggcatccaagatcttgtcaaccagaagtgcaagcaaaacattgcctgctgccagaactccccctccagcgcggatggcaaccttattggtgtcggtctcccttgcgttgcccttggctccatcctc

SEQ ID NO: 4 - protein, 157 aa, Aspergillus nidulans
  basf-rodA hydrophobin
MKFSIAAAVVAFAASVAALPPAHDSQFAGNGVGNKGNSNVKFPVPENVTVKQASDKCGDQAQLSCCNKATYAGDTTTVDEGLLSGALSGLIGAGSGAEGLGLFDQCSKLDVAVLIGIQDLVNQKCKQNIACCQNSPSSADGNLIGVGLPCVALGSIL

SEQ ID NO: 5 - DNA, 336 bp, Artificial Sequence
  CDS (1)..(336): basf-hypA from chemically synthesized polynucleotide
atgatctctcgcgtccttgtcgctgctctcgtcgctctccccgctcttgttactgcaactcctgctcccggaaagcctaaagccagcagtcagtgcgacgtcggtgaaatccattgctgtgacactcagcagactcccgaccacaccagcgccgccgcgtctggtttgcttggtgttcccatcaaccttggtgctttcctcggtttcgactgtacccccatttccgtccttggcgtcggtggcaacaactgtgctgctcagcctgtctgctgcacaggaaatcaattcaccgcattgattaacgctcttgactgctctcctgtcaatgtcaacctc

SEQ ID NO: 6 - protein, 112 aa, Artificial Sequence
  basf-hypA from chemically synthesized polynucleotide
MISRVLVAALVALPALVTATPAPGKPKASSQCDVGEIHCCDTQQTPDHTSAAASGLLGVPINLGAFLGFDCTPISVLGVGGNNCAAQPVCCTGNQFTALINALDCSPVNVNL

SEQ ID NO: 7 - DNA, 357 bp, Artificial Sequence
  CDS (1)..(357): basf-hypB from chemically synthesized polynucleotide
atggtcagcacgttcatcactgtcgcaaagacccttctcgtcgcgctcctcttcgtcaatatcaatatcgtcgttggtactgcaactaccggcaagcattgtagcaccggtcctatcgagtgctgcaagcaggtcatggattctaagagccctcaggctacggagcttcttacgaagaatggccttggcctgggtgtccttgctggcgtgaagggtcttgttggcgcgaattgcagccctatcacggcaattggtattggctccggcagccaatgctctggccagaccgtttgctgccagaataataatttcaacggtgttgtcgctattggttgcactcccattaatgccaatgtg

SEQ ID NO: 8 - protein, 119 aa, Artificial Sequence
  basf-hypB from chemically synthesized polynucleotide
MVSTFITVAKTLLVALLFVNINIVVGTATTGKHCSTGPIECCKQVMDSKSPQATELLTKNGLGLGVLAGVKGLVGANCSPITAIGIGSGSQCSGQTVCCQNNNFNGVVAIGCTPINANV

SEQ ID NO: 9 - DNA, 408 bp, Schyzophyllum commune
  CDS (1)..(408): basf-sc3 hydrophobin, cDNA template
atgttcgcccgtctccccgtcgtgttcctctacgccttcgtcgcgttcggcgccctcgtcgctgccctcccaggtggccacccgggcacgaccacgccgccggttacgacgacggtgacggtgaccacgccgccctcgacgacgaccatcgccgccggtggcacgtgtactacggggtcgctctcttgctgcaaccaggttcaatcggcgagcagcagccctgttaccgccctcctcggcctgctcggcattgtcctcagcgacctcaacgttctcgttggcatcagctgctctcccctcactgtcatcggtgtcggaggcagcggctgttcggcgcagaccgtctgctgcgaaaacacccaattcaacgggctgatcaacatcggttgcacccccatcaacatcctc

SEQ ID NO: 10 - protein, 136 aa, Schyzophyllum commune
  basf-sc3 hydrophobin, cDNA template
MFARLPVVFLYAFVAFGALVAALPGGHPGTTTPPVTTTVTVTTPPSTTTIAAGGTCTTGSLSCCNQVQSASSSPVTALLGLLGIVLSDLNVLVGISCSPLTVIGVGGSGCSAQTVCCENTQFNGLINIGCTPINIL

SEQ ID NO: 11 - DNA, 483 bp, Artificial Sequence
  CDS (1)..(483): basf-BASF1 from chemically synthesized polynucleotide
atgaagttctccgtctccgccgccgtcctcgccttcgccgcctccgtcgccgccctccctcagcacgactccgccgccggcaacggcaacggcgtcggcaacaagttccctgtccctgacgacgtcaccgtcaagcaggccaccgacaagtgcggcgaccaggcccagctctcctgctgcaacaaggccacctacgccggcgacgtcctcaccgacatcgacgagggcatcctcgccggcctcctcaagaacctcatcggcggcggctccggctccgagggcctcggcctcttcgaccagtgcgtcaagctcgacctccagatctccgtcatcggcatccctatccaggacctcctcaaccaggtcaacaagcagtgcaagcagaacatcgcctgctgccagaactccccttccgacgccaccggctccctcgtcaacctcggcctcggcaacccttgcatccctgtctccctcctccatatg

SEQ ID NO: 12 - protein, 161 aa, Artificial Sequence
  basf-BASF1 from chemically synthesized polynucleotide
MKFSVSAAVLAFAASVAALPQHDSAAGNGNGVGNKFPVPDDVTVKQATDKCGDQAQLSCCNKATYAGDVLTDIDEGILAGLLKNLIGGGSGSEGLGLFDQCVKLDLQISVIGIPIQDLLNQVNKQCKQNIACCQNSPSDATGSLVNLGLGNPCIPVSLLHM

SEQ ID NO: 13 - DNA, 465 bp, Artificial Sequence
  CDS (1)..(465): basf-BASF2 from chemically synthesized polynucleotide
atgaagttctccgtctccgccgccgtcctcgccttcgccgcctccgtcgccgccctccctcagcacgactccgccgccggcaacggcaacggcgtcggcaacaagttccctgtccctgacgacgtcaccgtcaagcaggccaccgacaagtgcggcgaccaggcccagctctcctgctgcaacaaggccacctacgccggcgacgtcaccgacatcgacgagggcatcctcgccggcctcctcaagaacctcatcggcggcggctccggctccgagggcctcggcctcttcgaccagtgcgtcaagctcgacctccagatctccgtcatcggcatccctatccaggacctcctcaaccagcagtgcaagcagaacatcgcctgctgccagaactccccttccgacgccaccggctccctcgtcaacctcggcaacccttgcatccctgtctccctcctccatatg

SEQ ID NO: 14 - protein, 155 aa, Artificial Sequence
  basf-BASF2 from chemically synthesized polynucleotide
MKFSVSAAVLAFAASVAALPQHDSAAGNGNGVGNKFPVPDDVTVKQATDKCGDQAQLSCCNKATYAGDVTDIDEGILAGLLKNLIGGGSGSEGLGLFDQCVKLDLQISVIGIPIQDLLNQQCKQNIACCQNSPSDATGSLVNLGNPCIPVSLLHM

SEQ ID NO: 15 - DNA, 882 bp, Bacillus subtilis
  CDS (1)..(882): basf-yaad yaaD
atggctcaaacaggtactgaacgtgtaaaacgcggaatggcagaaatgcaaaaaggcggcgtcatcatggacgtcatcaatgcggaacaagcgaaaatcgctgaagaagctggagctgtcgctgtaatggcgctagaacgtgtgccagcagatattcgcgcggctggaggagttgcccgtatggctgaccctacaatcgtggaagaagtaatgaatgcagtatctatcccggtaatggcaaaagcgcgtatcggacatattgttgaagcgcgtgtgcttgaagctatgggtgttgactatattgatgaaagtgaagttctgacgccggctgacgaagaatttcatttaaataaaaatgaatacacagttccttttgtctgtggctgccgtgatcttggtgaagcaacacgccgtattgcggaaggtgcttctatgcttcgcacaaaaggtgagcctggaacaggtaatattgttgaggctgttcgccatatgcgtaaagttaacgctcaagtgcgcaaagtagttgcgatgagtgaggatgagctaatgacagaagcgaaaaacctaggtgctccttacgagcttcttcttcaaattaaaaaagacggcaagcttcctgtcgttaactttgccgctggcggcgtagcaactccagctgatgctgctctcatgatgcagcttggtgctgacggagtatttgttggttctggtatttttaaatcagacaaccctgctaaatttgcgaaagcaattgtggaagcaacaactcactttactgattacaaattaatcgctgagttgtcaaaagagcttggtactgcaatgaaagggattgaaatctcaaacttacttccagaacagcgtatgcaagaacgcggctgg

SEQ ID NO: 16 - protein, 294 aa, Bacillus subtilis
  basf-yaad yaaD
MAQTGTERVKRGMAEMQKGGVIMDVINAEQAKIAEEAGAVAVMALERVPADIRAAGGVARMADPTIVEEVMNAVSIPVMAKARIGHIVEARVLEAMGVDYIDESEVLTPADEEFHLNKNEYTVPFVCGCRDLGEATRRIAEGASMLRTKGEPGTGNIVEAVRHMRKVNAQVRKVVAMSEDELMTEAKNLGAPYELLLQIKKDGKLPVVNFAAGGVATPADAALMMQLGADGVFVGSGIFKSDNPAKFAKAIVEATTHFTDYKLIAELSKELGTAMKGIEISNLLPEQRMQERGW

SEQ ID NO: 17 - DNA, 591 bp, Bacillus subtilis
  CDS (1)..(591): basf-yaae yaaE with Gly insert at position 2
atgggattaacaataggtgtactaggacttcaaggagcagttagagagcacatccatgcgattgaagcatgcggcgcggctggtcttgtcgtaaaacgtccggagcagctgaacgaagttgacgggttgattttgccgggcggtgagagcacgacgatgcgccgtttgatcgatacgtatcaattcatggagccgcttcgtgaattcgctgctcagggcaaaccgatgtttggaacatgtgccggattaattatattagcaaaagaaattgccggttcagataatcctcatttaggtcttctgaatgtggttgtagaacgtaattcatttggccggcaggttgacagctttgaagctgatttaacaattaaaggcttggacgagccttttactggggtattcatccgtgctccgcatattttagaagctggtgaaaatgttgaagttctatcggagcataatggtcgtattgtagccgcgaaacaggggcaattccttggctgctcattccatccggagctgacagaagatcaccgagtgacgcagctgtttgttgaaatggttgaggaatataagcaaaaggcacttgta

SEQ ID NO: 18 - protein, 197 aa, Bacillus subtilis
  basf-yaae yaaE with Gly insert at position 2
MGLTIGVLGLQGAVREHIHAIEACGAAGLVVKRPEQLNEVDGLILPGGESTTMRRLIDTYQFMEPLREFAAQGKPMFGTCAGLIILAKEIAGSDNPHLGLLNVVVERNSFGRQVDSFEADLTIKGLDEPFTGVFIRAPHILEAGENVEVLSEHNGRIVAAKQGQFLGCSFHPELTEDHRVTQLFVEMVEEYKQKALV

SEQ ID NO: 19 - DNA, 1329 bp, Artificial Sequence
  CDS (1)..(1329): basf-yaad-Xa-dewA-his fusion of Bacillus subtilis yaaD and N-terminal factor Xa proteinase cleavage site and Aspergillus nidulans hydrophobin dewA and his6
atggctcaaacaggtactgaacgtgtaaaacgcggaatggcagaaatgcaaaaaggcggcgtcatcatggacgtcatcaatgcggaacaagcgaaaatcgctgaagaagctggagctgtcgctgtaatggcgctagaacgtgtgccagcagatattcgcgcggctggaggagttgcccgtatggctgaccctacaatcgtggaagaagtaatgaatgcagtatctatcccggtaatggcaaaagcgcgtatcggacatattgttgaagcgcgtgtgcttgaagctatgggtgttgactatattgatgaaagtgaagttctgacgccggctgacgaagaatttcatttaaataaaaatgaatacacagttccttttgtctgtggctgccgtgatcttggtgaagcaacacgccgtattgcggaaggtgcttctatgcttcgcacaaaaggtgagcctggaacaggtaatattgttgaggctgttcgccatatgcgtaaagttaacgctcaagtgcgcaaagtagttgcgatgagtgaggatgagctaatgacagaagcgaaaaacctaggtgctccttacgagcttcttcttcaaattaaaaaagacggcaagcttcctgtcgttaactttgccgctggcggcgtagcaactccagctgatgctgctctcatgatgcagcttggtgctgacggagtatttgttggttctggtatttttaaatcagacaaccctgctaaatttgcgaaagcaattgtggaagcaacaactcactttactgattacaaattaatcgctgagttgtcaaaagagcttggtactgcaatgaaagggattgaaatctcaaacttacttccagaacagcgtatgcaagaacgcggctggagatccattgaaggccgcatgcgcttcatcgtctctctcctcgccttcactgccgcggccaccgcgaccgccctcccggcctctgccgcaaagaacgcgaagctggccacctcggcggccttcgccaagcaggctgaaggcaccacctgcaatgtcggctcgatcgcttgctgcaactcccccgctgagaccaacaacgacagtctgttgagcggtctgctcggtgctggccttctcaacgggctctcgggcaacactggcagcgcctgcgccaaggcgagcttgattgaccagctgggtctgctcgctctcgtcgaccacactgaggaaggccccgtctgcaagaacatcgtcgcttgctgccctgagggaaccaccaactgtgttgccgtcgacaacgctggcgctggtaccaaggctgagggatctcatcaccatcaccatcac

SEQ ID NO: 20 - protein, 443 aa, Artificial Sequence
  basf-yaad-Xa-dewA-his fusion of Bacillus subtilis yaaD and N-terminal factor Xa proteinase cleavage site and Aspergillus nidulans hydrophobin dewA and his6
MAQTGTERVKRGMAEMQKGGVIMDVINAEQAKIAEEAGAVAVMALERVPADIRAAGGVARMADPTIVEEVMNAVSIPVMAKARIGHIVEARVLEAMGVDYIDESEVLTPADEEFHLNKNEYTVPFVCGCRDLGEATRRIAEGASMLRTKGEPGTGNIVEAVRHMRKVNAQVRKVVAMSEDELMTEAKNLGAPYELLLQIKKDGKLPVVNFAAGGVATPADAALMMQLGADGVFVGSGIFKSDNPAKFAKAIVEATTHFTDYKLIAELSKELGTAMKGIEISNLLPEQRMQERGWRSIEGRMRFIVSLLAFTAAATATALPASAAKNAKLATSAAFAKQAEGTTCNVGSIACCNSPAETNNDSLLSGLLGAGLLNGLSGNTGSACAKASLIDQLGLLALVDHTEEGPVCKNIVACCPEGTTNCVAVDNAGAGTKAEGSHHHHHH

SEQ ID NO: 21 - DNA, 1395 bp, Artificial Sequence
  CDS (1)..(1395): basf-yaad-Xa-rodA-his fusion of Bacillus subtilis yaaD and N-terminal factor Xa proteinase cleavage site and Aspergillus nidulans hydrophobin rodA and his6
atggctcaaacaggtactgaacgtgtaaaacgcggaatggcagaaatgcaaaaaggcggcgtcatcatggacgtcatcaatgcggaacaagcgaaaatcgctgaagaagctggagctgtcgctgtaatggcgctagaacgtgtgccagcagatattcgcgcggctggaggagttgcccgtatggctgaccctacaatcgtggaagaagtaatgaatgcagtatctatcccggtaatggcaaaagcgcgtatcggacatattgttgaagcgcgtgtgcttgaagctatgggtgttgactatattgatgaaagtgaagttctgacgccggctgacgaagaatttcatttaaataaaaatgaatacacagttccttttgtctgtggctgccgtgatcttggtgaagcaacacgccgtattgcggaaggtgcttctatgcttcgcacaaaaggtgagcctggaacaggtaatattgttgaggctgttcgccatatgcgtaaagttaacgctcaagtgcgcaaagtagttgcgatgagtgaggatgagctaatgacagaagcgaaaaacctaggtgctccttacgagcttcttcttcaaattaaaaaagacggcaagcttcctgtcgttaactttgccgctggcggcgtagcaactccagctgatgctgctctcatgatgcagcttggtgctgacggagtatttgttggttctggtatttttaaatcagacaaccctgctaaatttgcgaaagcaattgtggaagcaacaactcactttactgattacaaattaatcgctgagttgtcaaaagagcttggtactgcaatgaaagggattgaaatctcaaacttacttccagaacagcgtatgcaagaacgcggctggagatctattgaaggccgcatgaagttctccattgctgccgctgtcgttgctttcgccgcctccgtcgcggccctccctcctgcccatgattcccagttcgctggcaatggtgttggcaacaagggcaacagcaacgtcaagttccctgtccccgaaaacgtgaccgtcaagcaggcctccgacaagtgcggtgaccaggcccagctctcttgctgcaacaaggccacgtacgccggtgacaccacaaccgttgatgagggtcttctgtctggtgccctcagcggcctcatcggcgccgggtctggtgccgaaggtcttggtctcttcgatcagtgctccaagcttgatgttgctgtcctcattggcatccaagatcttgtcaaccagaagtgcaagcaaaacattgcctgctgccagaactccccctccagcgcggatggcaaccttattggtgtcggtctcccttgcgttgcccttggctccatcctcggatctcatcaccatcaccatcac

SEQ ID NO: 22 - protein, 465 aa, Artificial Sequence
  basf-yaad-Xa-rodA-his fusion of Bacillus subtilis yaaD and N-terminal factor Xa proteinase cleavage site and Aspergillus nidulans hydrophobin rodA and his6
MAQTGTERVKRGMAEMQKGGVIMDVINAEQAKIAEEAGAVAVMALERVPADIRAAGGVARMADPTIVEEVMNAVSIPVMAKARIGHIVEARVLEAMGVDYIDESEVLTPADEEFHLNKNEYTVPFVCGCRDLGEATRRIAEGASMLRTKGEPGTGNIVEAVRHMRKVNAQVRKVVAMSEDELMTEAKNLGAPYELLLQIKKDGKLPVVNFAAGGVATPADAALMMQLGADGVFVGSGIFKSDNPAKFAKAIVEATTHFTDYKLIAELSKELGTAMKGIEISNLLPEQRMQERGWRSIEGRMKFSIAAAVVAFAASVAALPPAHDSQFAGNGVGNKGNSNVKFPVPENVTVKQASDKCGDQAQLSCCNKATYAGDTTTVDEGLLSGALSGLIGAGSGAEGLGLFDQCSKLDVAVLIGIQDLVNQKCKQNIACCQNSPSSADGNLIGVGLPCVALGSILGSHHHHHH

SEQ ID NO: 23 - DNA, 1407 bp, Artificial Sequence
  CDS (1)..(1407): basf-yaad-Xa-BASF1-his fusion of Bacillus subtilis yaaD and N-terminal factor Xa proteinase cleavage site and artificial hydrophobin; BASF1  BASF1 from chemically synthesized polynucleotide
atggctcaaacaggtactgaacgtgtaaaacgcggaatggcagaaatgcaaaaaggcggcgtcatcatggacgtcatcaatgcggaacaagcgaaaatcgctgaagaagctggagctgtcgctgtaatggcgctagaacgtgtgccagcagatattcgcgcggctggaggagttgcccgtatggctgaccctacaatcgtggaagaagtaatgaatgcagtatctatcccggtaatggcaaaagcgcgtatcggacatattgttgaagcgcgtgtgcttgaagctatgggtgttgactatattgatgaaagtgaagttctgacgccggctgacgaagaatttcatttaaataaaaatgaatacacagttccttttgtctgtggctgccgtgatcttggtgaagcaacacgccgtattgcggaaggtgcttctatgcttcgcacaaaaggtgagcctggaacaggtaatattgttgaggctgttcgccatatgcgtaaagttaacgctcaagtgcgcaaagtagttgcgatgagtgaggatgagctaatgacagaagcgaaaaacctaggtgctccttacgagcttcttcttcaaattaaaaaagacggcaagcttcctgtcgttaactttgccgctggcggcgtagcaactccagctgatgctgctctcatgatgcagcttggtgctgacggagtatttgttggttctggtatttttaaatcagacaaccctgctaaatttgcgaaagcaattgtggaagcaacaactcactttactgattacaaattaatcgctgagttgtcaaaagagcttggtactgcaatgaaagggattgaaatctcaaacttacttccagaacagcgtatgcaagaacgcggctggagatctattgaaggccgcatgaagttctccgtctccgccgccgtcctcgccttcgccgcctccgtcgccgccctccctcagcacgactccgccgccggcaacggcaacggcgtcggcaacaagttccctgtccctgacgacgtcaccgtcaagcaggccaccgacaagtgcggcgaccaggcccagctctcctgctgcaacaaggccacctacgccggcgacgtcctcaccgacatcgacgagggcatcctcgccggcctcctcaagaacctcatcggcggcggctccggctccgagggcctcggcctcttcgaccagtgcgtcaagctcgacctccagatctccgtcatcggcatccctatccaggacctcctcaaccaggtcaacaagcagtgcaagcagaacatcgcctgctgccagaactccccttccgacgccaccggctccctcgtcaacctcggcctcggcaacccttgcatccctgtctccctcctccatatgggatctcatcaccatcaccatcac

SEQ ID NO: 24 - protein, 469 aa, Artificial Sequence
  basf-yaad-Xa-BASF1-his fusion of Bacillus subtilis yaaD and N-terminal factor Xa proteinase cleavage site and artificial hydrophobin BASF1; BASF1 from chemically synthesized polynucleotide
MAQTGTERVKRGMAEMQKGGVIMDVINAEQAKIAEEAGAVAVMALERVPADIRAAGGVARMADPTIVEEVMNAVSIPVMAKARIGHIVEARVLEAMGVDYIDESEVLTPADEEFHLNKNEYTVPFVCGCRDLGEATRRIAEGASMLRTKGEPGTGNIVEAVRHMRKVNAQVRKVVAMSEDELMTEAKNLGAPYELLLQIKKDGKLPVVNFAAGGVATPADAALMMQLGADGVFVGSGIFKSDNPAKFAKAIVEATTHFTDYKLIAELSKELGTAMKGIEISNLLPEQRMQERGWRSIEGRMKFSVSAAVLAFAASVAALPQHDSAAGNGNGVGNKFPVPDDVTVKQATDKCGDQAQLSCCNKATYAGDVLTDIDEGILAGLLKNLIGGGSGSEGLGLFDQCVKLDLQISVIGIPIQDLLNQVNKQCKQNIACCQNSPSDATGSLVNLGLGNPCIPVSLLHMGSHHHHHH

SEQ ID NO: 25 - DNA, 28 bp, Artificial Sequence
  Chemically synthesized Hal570 primer
gcgcgcccatggctcaaacaggtactga

SEQ ID NO: 26 - DNA, 28 bp, Artificial Sequence
  Chemically synthesized Hal571 primer
gcagatctccagccgcgttcttgcatac

SEQ ID NO: 27 - DNA, 30 bp, Artificial Sequence
  Chemically synthesized Hal572 primer
ggccatgggattaacaataggtgtactagg

SEQ ID NO: 28 - DNA, 33 bp, Artificial Sequence
  Chemically synthesized Hal573 primer
gcagatcttacaagtgccttttgcttatattcc

SEQ ID NO: 29 - DNA, 38 bp, Artificial Sequence
  Chemically synthesized KaM416 primer
gcagcccatcagggatccctcagccttggtaccagcgc

SEQ ID NO: 30 - DNA, 50 bp, Artificial Sequence
  Chemically synthesized KaM417 primer
cccgtagctagtggatccattgaaggccgcatgaagttctccgtctccgc

SEQ ID NO: 31 - DNA, 45 bp, Artificial Sequence
  Chemically synthesized KaM434 primer
gctaagcggatccattgaaggccgcatgaagttctccattgctgc

SEQ ID NO: 32 - DNA, 30 bp, Artificial Sequence
  Chemically synthesized KaM435 primer
ccaatggggatccgaggatggagccaaggg

SEQ ID NO: 33 - DNA, 38 bp, Artificial Sequence
  Chemically synthesized KaM418 primer
ctgccattcaggggatcccatatggaggagggagacag

SEQ ID NO: 34 - DNA, 32 bp, Artificial Sequence
  Chemically synthesized KaM464 primer
cgttaaggatccgaggatgttgatgggggtgc

SEQ ID NO: 35 - DNA, 35 bp, Artificial Sequence
  Chemically synthesized KaM465 primer
gctaacagatctatgttcgcccgtctccccgtcgt

(19) United States
(12) Patent Application Publication
(10) Pub. No.: US 2010/0267096 A1
(43) Pub. Date: Oct. 21, 2010

(54) USE OF A SYNERGISTIC MIXTURE OF WATER-SOLUBLE POLYMERS AND HYDROPHOBINS FOR THICKENING AQUEOUS PHASES

(75) Inventors: Ulf Baus, Dossenheim (DE); Thorsten Montag, Dudenhofen (DE); Thomas Subkowski, Ladenburg (DE); Claus Bollschweiler, Heidelberg (DE); Kati Schmidt, Ludwigshafen (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/719,900

(22) Filed: Mar. 9, 2010

(30) Foreign Application Priority Data
Mar. 9, 2009 (EP) 09154643.2

(51) Int. Cl. C08L 89/00; C08L 5/00; C12P 13/02

(52) U.S. Cl. 435/129; 524/17; 524/18

(57) ABSTRACT

The invention relates to the use of a synergistic mixture of water-soluble polymers with thickening action and hydrophobins for thickening aqueous phases, and to the degradation of the thickening action by cleaving the protein. The invention further relates to a thickening composition of water-soluble polymers, hydrophobins and water.

### RELATED APPLICATIONS

This application claims benefit of European application 09154643.2, filed Mar. 9, 2009, which is incorporated by reference herein in its entirety for all useful purposes.

### SUBMISSION OF SEQUENCE LISTING

The Sequence Listing associated with this application is filed in electronic format via EFS-Web and hereby incorporated by reference into the specification in its entirety. The name of the text file containing the Sequence Listing is Sequence_Listing_{—}12810_{—}00993_US.txt. The size of the text file is 70.5 KB, and the text file was created on Mar. 8, 2010.

### BACKGROUND OF THE INVENTION

The present invention relates to the use of a synergistic mixture of water-soluble polymers with thickening action and hydrophobins for thickening aqueous phases, and to the degradation of the thickening action by cleaving the protein. The present invention further relates to a thickening composition of water-soluble polymers, hydrophobins and water.

Water-soluble polymers with thickening action are used in many fields of industry, for example in the cosmetics sector, in foods, for production of cleaning compositions, printing inks, emulsion paints or in mineral oil extraction.

Polymers with thickening action used are a multitude of chemically different polymers, for example biopolymers such as xanthan, starch, gelatin, modified biopolymers such as hydroxyethylcellulose, hydroxypropylcellulose or carboxymethylcellulose, or synthetic polymers such as polyvinyl alcohols, polyacrylic acids or partly crosslinked polyacrylic acids, or polyacrylamides, and especially copolymers of (meth)acrylic acid with further monomers.

A further class of polymers with thickening action is that of the so-called associative thickeners. These are water-soluble polymers which have lateral or terminal hydrophobic groups, for example relatively long alkyl chains. In aqueous solution, such hydrophobic groups may associate with themselves or with other substances having hydrophobic groups. This forms an associative network, through which the medium is thickened. Examples of such polymers are disclosed in EP 013 836 A1 or U.S. Patent Publication 2008/0103248.

Hydrophobins are small proteins of about 100 to 150 amino acids, which are characteristic of filamentous fungi, for example Schizophyllum commune. They generally have 8 cysteine units. They form relatively mobile solutions in water at low concentrations of up to approx. 3% by weight, whereas more highly concentrated solutions finally become gelatinous.

### DESCRIPTION OF RELATED ART

The prior art has proposed the use of hydrophobins for various applications.

EP 1 252 516 discloses coating various substrates with a solution comprising hydrophobins at a temperature of 30° to 80° C. In addition, for example, use as a demulsifier (WO 2006/103251), as an evaporation retardant (WO 2006/128877) or soiling inhibitor (U.S. Patent Publication 2009/0305930) was proposed.

U.S. Patent Publication 2009/0131281 discloses drilling muds which comprise hydrophobins. The formulations may comprise, in addition to the hydrophobins, a wide variety of different other components, including polymers or copolymers, for example polyacrylamides.

WO 96/41882 proposes the use of hydrophobins as emulsifiers, thickeners and surface-active substances for hydrophilizing hydrophobic surfaces, for improving the water stability of hydrophilic substrates, for production of oil-in-water emulsions or of water-in-oil emulsions. Additionally proposed are pharmaceutical applications such as the production of ointments or creams, and cosmetic applications such as skin protection or the production of shampoos or hair rinses.

However, no document discloses that a mixture of hydrophobins with water-soluble polymers having thickening action in a weight ratio of 5:1 to 1:10 has synergistic effects.

### BRIEF DESCRIPTION OF THE FIGURE

FIG. 1 shows the viscosities of solutions of polymer A1 at pH 9 as a function of time (curve 1: only 1.2% polymer; curve 2:1% polymer+0.5% hydrophobin A; curve 3:1% polymer+0.5% hydrophobin B). A clear time dependence of the viscosity of the mixtures of hydrophobin and polymer Al is discerned, while polymer A1 alone has no time dependence.

### DETAILED DESCRIPTION OF THE INVENTION

For some applications of thickening polymers, it is desired that the thickening action can be reversed. A typical example of this is the “fracturing” process in the course of mineral oil production. This involves injecting a solution of a thickening polymer into a borehole. This pressure treatment forms new fissures in the mineral oil formation, through which the mineral oil flows better out of the formation into the borehole. After the “fracturing” has ended, the viscosity of the polymer solution should, however, be degraded again, so that the polymer solution does not block the fissures formed. For degradation of the polymers, for example, the use of oxidizing agents has been proposed. In the case of biopolymers such as polysaccharides, degradation using enzymes is also known, the enzymes breaking the polymer chain at particular sites. Such a process has been proposed, for example, by U.S. Pat. No. 5,201,370. Since enzymes are generally relatively selective, it is also necessary to stock other enzymes for cleavage of other biopolymers, while synthetic polymers generally cannot be cleaved by enzymes at all.

It was an object of the invention to provide a composition with thickening action, in which the thickening action can be “switched off” again in a simple manner.

It has been found, surprisingly, that hydrophobins and water-soluble polymers interact synergistically and, even in low concentrations, form compositions with good thickening action. The thickening action can, if desired, be eliminated in a simple manner by cleaving the hydrophobin, for example with the aid of enzymes. Cleavage of the thickening polymer itself is not required.

Accordingly, we have found the use of a synergistic mixture for thickening aqueous phases, the mixture comprising
at least one water-soluble polymer (A) with thickening action, and
at least one hydrophobin (B),
   in a weight ratio (A)/(B) of 5:1 to 1:10.

We additionally have found a synergistic composition that comprises at least
an aqueous phase,
0.01% to 2.5% by weight of at least one water-soluble polymer (A) with thickening action, and
0.1% to 2.5% by weight of at least one hydrophobin (B),
   wherein the weight ratio (A)/(B) is 5:1 to 1:10, and where the amounts stated are based on the sum of all components of the aqueous phase.

With regard to the invention, the following can be stated specifically:

Thickening Polymer (A)

According to the invention, at least one water-soluble thickening polymer (A) is used for thickening.

It will be appreciated that the term “polymer” also comprises copolymers of two or more monomers. Suitable water-soluble thickening polymers (A) generally have a number-average molar mass Mₙ of 1000 to 10,000,000 g/mol, preferably 10,000 to 1,000,000 g/mol.

The polymers (A) used may be miscible with water without a miscibility gap, without this being absolutely necessary for performance of the invention. However, they must dissolve in water at least to such a degree that the inventive use is possible. In general, the polymers (A) used must have a solubility in water of at least 50 g/l, preferably 100 g/1 and more preferably at least 200 g/l.

The person skilled in the art in the field of thickening polymers is aware that the solubility of thickening polymers in water may depend on the pH. The reference point for the assessment of the water solubility in each case should therefore be the pH desired for the particular end use of the thickening mixture. A polymer (A) that has insufficient solubility for the intended end use at a particular pH may have sufficient solubility at another pH. The term “water-soluble” is thus also based, for example, on alkali-soluble emulsions (ASE) of polymers.

The term “thickening polymer” is used in this invention in a manner known in principle for those polymers which, even in comparatively small concentrations, significantly increase the viscosity of aqueous solutions.

Suitable water-soluble thickening polymers (A) comprise, as well as carbon and hydrogen, hydrophilic groups in such an amount that the polymers (A) become water-soluble, at least within particular pH ranges. More particularly, these are functional groups which comprise oxygen and/or nitrogen atoms. The oxygen and/or nitrogen atoms may be part of the main chain of the polymer and/or may be arranged laterally or terminally. Examples of suitable functional groups are carbonyl groups>C═O, ether groups —O—, especially polyethylene oxide groups —(CH₂—CH₂—O—)ₙ— where n is preferably from 1 to 200, hydroxyl groups 13 OH, ester groups —C(O)O—, primary, secondary or tertiary amino groups, amide groups —C(O)—NH—, carboxamide groups —C(O)—NH₂, urea groups —NH—C(O)—NH—, urethane groups —O—C(O)—NH— or acidic groups such as carboxyl groups —COOH, sulfonic acid groups —SO₃H, phosphonic acid groups —PO₃H₂ or phosphoric acid groups —OP(OH)₃.

Examples of preferred functional groups are hydroxyl groups —OH, carboxyl groups —COOH, sulfonic acid groups —SO₃H, carboxamide groups —C(O)—NH₂ and polyethylene oxide groups —(CH₂—CH₂—O—)ₙ— where n preferably is an integer from 1 to 200.

Water-soluble thickening polymers (A) suitable for performance of the invention generally have a numerical ratio of oxygen and nitrogen atoms to the total number of oxygen and nitrogen and carbon atoms, (n_{O}+n_{N})/(n_{C}+n_{O}+n_{N}), of 0.2 to 0.5, preferably 0.3 to 0.46.

The thickening polymers may comprise naturally occurring polymers, modified natural polymers or synthetic polymers.

Naturally occurring thickening polymers may comprise, for example, polypeptides such as gelatin or casein.

Naturally occurring thickening polymers may also be polysaccharides, which term shall also comprises modified polysaccharides. Examples of polysaccharides are starch, xanthans or glucans. Examples of modified polysaccharides are hydroxyethylcellulose, hydroxypropylcellulose, hydroxypropylmethylcellulose or carboxymethylcellulose. It is possible with preference to use xanthans or glucans.

Examples of synthetic polymers are poly(meth)acrylic acid and salts thereof, copolymers comprising poly(meth)acrylic acid and salts thereof, polyacrylamides, polyvinylpyrrolidone, polyvinyl alcohol or polyethylene glycols. Examples of synthetic polymers may also be crosslinked poly(meth)acrylic acids or poly(meth)acrylic acid copolymers, provided that the crosslinking is not so great that it impairs the water solubility of the polymers.

The polyacrylic acids may be solutions of polyacrylic acid or copolymers thereof, or precipitation polymers based on polyacrylic acid, which also can be crosslinked easily.

Further examples are alkali-soluble emulsions of (meth)acrylic acid copolymers. Such copolymers are present in the acidic pH range as comparatively mobile emulsions in water. In the alkaline range, the polymers dissolve in the aqueous phase and increase the viscosity thereof significantly. Alkali-soluble emulsions are, for example, copolymers that comprise (meth)acrylic acid and hydrophobic comonomers, especially (meth)acrylic esters, especially C₁- to C₄-alkyl(meth)acrylates, such as methyl(meth)acrylate, ethyl(meth)acrylate or n-butyl(meth)acrylate. The amount of (meth)acrylic acid is typically 10% to 50% by weight, and the amount of further co-monomers, especially of the (meth)acrylates, 50% to 90% by weight.

They may also be hydrophobically associative polymers. In a manner known in principle, these are understood to mean water-soluble polymers that have lateral or terminal hydrophobic groups, for example, relatively long alkyl chains. In aqueous solution, such hydrophobic groups may associate with themselves or with substances having other hydrophobic groups, which cause significant thickening action.

Examples of preferred hydrophobically associative polymers are copolymers that comprise acidic monomers, preferably (meth)acrylic acid and at least one (meth)acrylic ester, where the ester group comprises a hydrocarbon radical R¹ with at least 6 carbon atoms, preferably 8 to 30 carbon atoms. These may preferably be linear aliphatic hydrocarbon radicals or hydrocarbon radicals comprising aromatic units, especially ω-aryl-substituted alkyl radicals. The (meth)acrylic esters may be simple esters of the formula H₂C═C(R²)—COOR¹ where R² may be H or CH₃. The hydrocarbon radical R¹ is preferably bonded via a hydrophilic spacer to the (meth)acrylic acid radical, i.e. it is a (meth)acrylic ester of the general formula H₂C═C(R²)—COO—R³—R¹ where R³ is a divalent hydrophilic group. R³ is preferably a polyalkylene oxide group —(CH₂—CH(R⁴)—O—)ₙ— where n is an integer from 2 to 100, preferably 5 to 50, and R⁴ is independently H or CH₃, provided that at least 50 mol %, preferably at least 80 mol %, of the R⁴ radicals are H. R⁴ is preferably and exclusively H.

The amount of the H₂C═C(R²)—COO—R³—R¹ monomers is typically 1% to 20% by weight based on the sum of all monomers. The further monomers may exclusively be (meth)acrylic acid. In addition, further (meth)acrylic esters may be present, especially C₁- to C₄-alkyl(meth)acrylates, such as methyl(meth)acrylate, ethyl(meth)acrylate or n-butyl(meth)acrylate. For example, they may be polymers which comprise 1 to 20% by weight, preferably 5 to 15% by weight, of H₂C═C(R²)COO—(CH₂—CH(R⁴)—O—)ₙ—R¹, 10% to 80% by weight, preferably 20% to 80% by weight, of (meth)acrylic acid and 5% to 70% by weight, preferably 10% to 65% by weight, of C₁- to C₄-alkyl(meth)acrylates, each of the amounts being based on all monomers in the polymer. This makes it possible to obtain alkali-free emulsions which additionally possess hydrophobically associative groups.

Further examples of hydrophobically associative polymers are hydrophobically modified cellulose ethers, hydrophobically modified polyacrylamides, hydrophobically modified polyethers, for example polyethylene glycol terminally capped with C₆- to C₃₀-hydrocarbon groups, or hydrophobically associative polyurethanes which comprise polyether segments and terminal hydrophobic groups.

Hydrophobins (B)

According to the invention, at least one hydrophobin (B) is additionally used for thickening.

The term “hydrophobins” shall be understood hereinafter to mean polypeptides of the general structural formula (I)

Xₙ—C¹—X₁₋₅₀—C²—X₀₋₅—C³—X₁₋₁₀₀—C⁴—X₁₋₁₀₀—C⁵—X₁₋₁₅₀—C⁶—X₀₋₅—C⁷—X₁₋₅₀—C⁸Xₘ (I)

where X may be any of the 20 naturally occurring amino acids (Phe, Leu, Ser, Tyr, Cys, Trp,

Pro, His, Gln, Arg, Ile, Met, Thr, Asn, Lys, Val, Ala, Asp, Glu, Gly). In the formula (I), the X residues may be the same or different in each case. The indices beside X are each the number of amino acids in the particular part-sequence X, C is cysteine, alanine, serine, glycine, methionine or threonine, where at least four of the residues designated with C are cysteine, and the indices n and m are each independently natural numbers between 0 and 500, preferably between 15 and 300.

The polypeptides of the formula (I) are also characterized by the property that, at room temperature, after coating a glass surface, they bring about an increase in the contact angle of a water droplet of at least 20°, preferably at least 25° and more preferably 30°, compared in each case with the contact angle of an equally large water droplet with the uncoated glass surface.

The amino acids designated with C¹ to C⁸ are preferably cysteines. However, they also may be replaced by other amino acids of similar bulk, preferably by alanine, serine, threonine, methionine or glycine. However, at least four, preferably at least 5, more preferably at least 6 and in particular at least 7 of positions C¹ to C⁸ should consist of cysteines. In the inventive proteins, cysteines may be present either in reduced form or form disulfide bridges with one another. Particular preference is given to the intramolecular formation of C—C bridges, especially those with at least one intramolecular disulfide bridge, preferably 2, more preferably 3 and most preferably 4 intramolecular disulfide bridges. In the case of the above-described exchange of cysteines for amino acids with similar space-filling, such C positions are advantageously exchanged in pairs that can form intramolecular disulfide bridges with one another.

If cysteines, serines, alanines, glycines, methionines or threonines are also used in the positions designated with X, the numbering of the individual C positions in the general formulae can change correspondingly.

Preference is given to using hydrophobins of the general formula (II)

Xₙ—C¹—X₃₋₂₅—C²—X₀₋₂—C³—X₅₋₅₀—C⁴—X₂₋₃₅—C⁵—X₂₋₁₅—C⁶—X₀₋₂—C⁷—X₃₋₃₅—C⁸—Xₘ (II)

to perform the present invention, where X, C and the indices beside X and C are each as defined above, the indices n and m are each whole numbers between 0 and 350, preferably from 15 to 300, and the proteins additionally feature the above-illustrated change in contact angle, and, furthermore, at least 6 of the residues designated with C are cysteine. More preferably, all C residues are cysteine.

Particular preference is given to using hydrophobins of the general formula (III)

Xₙ—C¹—X₅₋₉—C²—C³—X₁₁₋₃₉—C⁴—X₂₋₂₃—C⁵—X₅₋₉—C⁶—C⁷—X₆₋₁₈—C⁸—Xₘ (III)

where X, C and the indices beside X are each as defined above, the indices n and m are each whole numbers between 0 and 200, and the proteins additionally feature the above-illustrated change in contact angle, and at least 6 of the residues designated with C are cysteine. More preferably, all C residues are cysteine.

The Xₙ and Xₘ residues may be peptide sequences that naturally are joined to a hydrophobin. However, one residue or both residues may also be peptide sequences that are not naturally joined to a hydrophobin. This is understood to mean those Xₙ and/or Xₘ residues in which a peptide sequence that occurs naturally in a hydrophobin is lengthened by a peptide sequence that does not occur naturally in a hydrophobin.

If Xₙ and/or Xₘ are peptide sequences that are not naturally bonded to hydrophobins, such sequences are generally at least 20, preferably at least 35 amino acids in length. They may, for example, be sequences of from 20 to 500, preferably from 30 to 400 and more preferably from 35 to 100 amino acids. Such a residue that is not joined naturally to a hydrophobin also will be referred to hereinafter as a fusion partner. This is intended to express that the proteins may consist of at least one hydrophobin moiety and a fusion partner moiety that does not occur together in this form in nature. Fusion hydrophobins composed of fusion partner and hydrophobin moiety are described, for example, in U.S. Patent Publications 2009/0104663, 2008/0319168 and 2009/0136996.

The fusion partner moiety may be selected from a multitude of proteins. It is possible for only one single fusion partner to be bonded to the hydrophobin moiety, or it is also possible for a plurality of fusion partners to be joined to one hydrophobin moiety, for example on the amino terminus (Xₙ) and on the carboxyl terminus (Xₘ) of the hydrophobin moiety. However, it is also possible, for example, for two fusion partners to be joined to one position (Xₙ or Xₘ) of the inventive protein.

Particularly suitable fusion partners are proteins which naturally occur in microorganisms, especially in E. coli or Bacillus subtilis. Examples of such fusion partners are the sequences yaad (SEQ ID NO:), yaae (SEQ ID NO: 18), ubiquitin and thioredoxin. Also very suitable are fragments or derivatives of these sequences which comprise only some, for example from 70 to 99%, preferentially from 5 to 50% and more preferably from 10 to 40% of the sequences mentioned, or in which individual amino acids or nucleotides have been changed compared to the sequence mentioned, in which case the percentages are each based on the number of amino acids.

In a further preferred embodiment, the fusion hydrophobin, as well as the fusion partner mentioned as one of the Xₙ or Xₘ groups or as a terminal constituent of such a group, also has a so-called affinity domain (affinity tag/affinity tail). In a manner known in principle, this comprises anchor groups that can interact with particular complementary groups and can serve for easier work-up and purification of the proteins. Examples of such affinity domains are (His)ₖ, (Arg)_{k,} (Asp)ₖ, (Phe)ₖ or (Cys)ₖ groups, where k is generally a natural number from 1 to 10. An affinity domain may preferably be a (His)ₖ group, where k is from 4 to 6. In this case, the Xₙ and/or Xₘ group may consist exclusively of such an affinity domai, or else an Xₙ or Xₘ residue that is or is not naturally bonded to a hydrophobin is extended by a terminal affinity domain.

The hydrophobins used in accordance with the invention may also be modified in their polypeptide sequence, for example by glycosylation, acetylation or by chemical crosslinking with, for example, glutaraldehyde.

One property of the hydrophobins or derivatives thereof used in accordance with the invention is the change in surface properties when surfaces are coated with the proteins. The change in the surface properties can be determined experimentally, for example, by measuring the contact angle of a water droplet before and after the coating of the surface with the hydrophobin and determining the difference of the two measurements.

The performance of contact angle measurements is known in principle to those skilled in the art. The measurements are based on room temperature water droplets of 5 μl and the use of glass plates as substrates. The exact experimental conditions for an example of a suitable method for measuring the contact angle are given in the experimental section. Under the conditions mentioned there, the fusion proteins used in accordance with the invention have the property of increasing the contact angle by at least 20°, preferably at least 25°, more preferably at least 30°, compared with the contact angle of an equally large water droplet on the uncoated glass surface.

Particularly preferred hydrophobins for performing the present invention are the hydrophobins of the dewA, rodA, hypA, hypB, sc3, basf1, basf2 type. These hydrophobins, including their sequences are disclosed, for example, in U.S. Patent Publication 2009/0104663. Unless stated otherwise, the sequences specified below are based on the sequences disclosed in U.S. Patent Publication 2009/0104663. An overview table with the SEQ ID NOs: can be found in U.S. Patent Publication 2009/0104663 at paragraph [0105]. Unless explicitly stated otherwise, all SEQ ID NOs: cited herein are the same as the SEQ ID NOs: disclosed in U.S. Patent Publication 2009/0104663.

Especially suitable in accordance with the invention are the fusion proteins yaad-Xa-dewA-his (SEQ ID NO: 20), yaad-Xa-rodA-his (SEQ ID NO: 22) or yaad-Xa-basf1-his (SEQ ID NO: 24), with the polypeptide sequences specified in brackets and the nucleic acid sequences which code therefor, especially the sequences according to SEQ ID NOs: 19, 21, 23. More preferably, yaad-Xa-dewA-his (SEQ ID NO: 20) can be used. Proteins that, proceeding from the polypeptide sequences shown in SEQ ID NOs: 20, 22 or 24, arise through exchange, insertion or deletion of from at least one up to 10, preferably 5, amino acids, more preferably 5% of all amino acids, and which still have the biological property of the starting proteins to an extent of at least 50%, are also particularly preferred embodiments. A biological property of the proteins is understood here to mean the change in the contact angle by at least 20° , which has already been described.

Derivatives particularly suitable for performing the present invention are derivatives derived from yaad-Xa-dewA-his (SEQ ID NO: 20), yaad-Xa-rodA-his (SEQ ID NO: 22) or yaad-Xa-basf1-his (SEQ ID NO: 24) by truncating the yaad fusion partner. Instead of the complete yaad fusion partner (SEQ ID NO: 16) with 294 amino acids, it may be advantageous to use a truncated yaad residue. The truncated residue should, though, comprise at least 20, more preferably at least 35, amino acids. For example, a truncated residue having from 20 to 293, preferably from 25 to 250, more preferably from 35 to 150 and, for example, from 35 to 100 amino acids may be used. One example of such a protein is yaad40-Xa-dewA-his (SEQ ID NO: 26), which has a yaad residue truncated by 40 amino acids.

A cleavage site between the hydrophobin and the fusion partner or the fusion partners can be utilized to split off the fusion partner and to release the pure hydrophobin in underivatized form (for example by BrCN cleavage at methionine, factor Xa cleavage, enterokinase cleavage, thrombin cleavage, TEV cleavage, etc.).

The hydrophobins used in accordance with the invention can be prepared chemically by known methods of peptide synthesis, for example by Merrifield solid-phase synthesis.

Naturally occurring hydrophobins can be isolated from natural sources by means of suitable methods. Reference is made by way of example to Wosten et. al., Eur. J Cell Bio. 63, 122-129 (1994) or WO 96/41882.

A recombinant production process for hydrophobins without fusion partners from Talaromyces thermophilus is described by U.S. Patent Publication 2006/0040349.

Fusion proteins can be prepared preferably by genetic engineering methods, in which one nucleic acid sequence, especially DNA sequence, encoding the fusion partner and one encoding the hydrophobin moiety are combined in such a way that the desired protein is generated in a host organism as a result of gene expression of the combined nucleic acid sequence. Such a preparation process is disclosed, for example, by U.S. Patent Publications 2009/0104663 or 2008/0319168. The fusion partners make the production of the hydrophobins considerably easier. Fusion hydrophobins are produced in recombinant methods with significantly better yields than hydrophobins without fusion partners.

The fusion hydrophobins produced by the recombinant method from the host organisms can be worked up in a manner known in principle and be purified by means of known chromatographic methods.

In a preferred embodiment, the simplified workup and purification method disclosed in U.S. Patent Publication 2008/0319168, page 5, can be used. For this purpose, the fermented cells are first removed from the fermentation broth and digested, and the cell fragments are separated from the inclusion bodies. The latter advantageously can be effected by centrifugation. Finally, the inclusion bodies can be digested in a manner known in principle, for example, by means of acids, bases and/or detergents, in order to release the fusion hydrophobins. The inclusion bodies comprising the fusion hydrophobins used in accordance with the invention can generally be dissolved completely within approx. 1 h. even using 0.1 M NaOH.

The resulting solutions can be used, if appropriate after establishing the desired pH, without further purification to perform this invention. The fusion hydrophobins can, however, also be isolated from the solutions as a solid. Preferably, the isolation can be effected by means of spray granulation or spray drying, as described in U.S. Patent Publication 2008/0319168, page 52. The products obtained after the simplified workup and purification method comprise, as well as residues of cell fragments, generally from approx. 80% to 90% by weight of proteins. Depending on the fusion construct and fermentation conditions, the amount of fusion hydrophobins is generally from 30% to 80% by weight based on the amount of all proteins.

The isolated products comprising fusion hydrophobins can be stored as solids and can be dissolved for use in the media desired in each case.

The fusion hydrophobins can be used as such or else, after detaching and removing the fusion partner, as “pure” hydrophobins for the performance of this invention. A cleavage advantageously is undertaken after the isolation of the inclusion bodies and their dissolution.

Use of a Mixture of (A) and (B) for Thickening Aqueous Phases

According to the invention, a combination of at least one water-soluble polymer (A) with thickening action and at least one hydrophobin (B) is used to thicken aqueous phases. It will be appreciated that it is also possible to use mixtures of a plurality of different polymers (A) and/or a plurality of different hydrophobins, provided that no undesired effects occur.

Aqueous phases comprise water or an aqueous solvent mixture. Further solvent components in an aqueous solvent mixture are water-miscible solvents, for example alcohols such as methanol, ethanol or propanol. The proportion of water in a solvent mixture is generally at least 75% by weight based on the sum of all solvents used, preferably at least 90% by weight, more preferably at least 95% by weight and most preferably exclusively water is used.

In addition, the aqueous phases may comprise further inorganic or organic components dissolved or dispersed therein. The type and amount of further components are guided by the type of aqueous phase.

The amount of all thickening polymers (A) together is determined by the person skilled in the art according to the desired viscosity of the composition. It may also depend on the type and the molar mass of the polymer (A) and the other components present in the aqueous phase to be thickened. The amount of polymer (A) to be used is generally 0.01% to 2.5% by weight based on the sum of all components of the composition, preferably 0.1% to 2% by weight, more preferably 0.25% to 1.5% by weight and, for example, 0.5% to 1% by weight.

The amount of the hydrophobins (B) is determined by the person skilled in the art according to the desired viscosity of the composition. It may also depend on the other components present in the aqueous phase to be thickened. The amount of the hydrophobin (B) to be used is generally 0.1% to 2.5% by weight based on the sum of all components of the aqueous phase, preferably 0.2% to 2% by weight and more preferably 0.25% to 1% by weight.

According to the invention, the water-soluble polymers (A) and the hydrophobins (B) are used in a weight ratio (A)/(B) of 5:1 to 1:10. The weight ratio (A)/(B) is preferably 3:1 to 1:2.

For the inventive use, the water-soluble polymers (A) and the hydrophobins (B) are added in the amounts and ratios specified for each to the aqueous phase to be thickened. In this context, components (A) and (B) are preferably each dissolved separately in water or an aqueous solvent mixture and each added separately with intensive mixing to the aqueous phase to be thickened. The thickening effect sets in with the mixing of components (A) and (B).

According to the type of polymer (A) and of the aqueous phase to be thickened, however, other procedures are also conceivable. In the case of polymers (A) which have the thickening effect only within a particular pH range, it is possible, for example, to mix the polymer (A) and the hydrophobin (B) with one another and to add them to the aqueous phase, and only thereafter to adjust the pH to the desired value, which establishes the desired viscosity.

By means of mixture of water-soluble polymers (A) with thickening action and hydrophobins (B), it is possible to thicken a wide variety of different aqueous phases. The aqueous phases may, for example, be aqueous washing and cleaning composition formulations, for example washing compositions, washing aids, for example. pre-spotters, fabric softeners, cosmetic formulations, pharmaceutical formulations, foods, coating slips, formulations for textile manufacture, textile printing pastes, printing inks, printing pastes for textile printing, paints, pigment slurries, aqueous formulations for foam generation, formulations for the construction industry, for example concrete mixtures, formulations for mineral oil extraction, for example, drilling muds or formulations for acidizing or fracturing, or deicing mixtures, for example for aircraft.

In the inventive mixture, after the thickening of the aqueous phase, the thickening action can optionally be degraded again. To this end, at least one agent capable of cleaving peptide bonds in the hydrophobin is added to the aqueous phase. The cleavage of the hydrophobin at least significantly reduces or even eliminates the thickening action according to the type of composition.

The cleavage can be effected by means of customary chemical agents; for example, it may be a BrCN cleavage. In a preferred embodiment, it is possible to use enzymes for selective cleavage of particular peptide bonds. In a particularly preferred embodiment of the invention, proteases are used to cleave the hydrophobins.

This embodiment can, for example, be used advantageously in the mineral oil extraction sector for treatment of underground mineral oil-bearing formations. To this end, a solution of the water-soluble polymer (A) and the hydrophobin (B) is injected into the mineral oil-bearing formation through a borehole. This pressure treatment forms new fissures in the mineral oil-bearing formation, through which the mineral oil can flow better out of the formation to the borehole. Such a treatment is also referred to as “fracturing”. After the end of the treatment, a solution comprising the agent which can cleave peptide bonds, preferably a protease solution, is injected into the formation. This cleaves the hydrophobins; the viscosity of the thickened aqueous phase decreases again. This advantageously prevents the thickened aqueous phase from blocking the newly formed fissures, thus negating the success of the fracturing treatment.

In a further example, an aircraft can first be deiced with a mixture thickened in accordance with the invention. After the deicing, the residues of the mixture can be treated with an agent that cleaves peptide bonds, preferably a protease solution, in order that the residues of the deicing mixture do not contaminate the airfield.

Synergistic Thickener Composition

In a further aspect, the invention relates to a synergistic composition that comprises at least one aqueous phase, 0.01% to 2.5% by weight of at least one water-soluble polymer (A) with thickening action, and at least 0.1% to 2.5% by weight of at least one hydrophobin (B), wherein the weight ratio (A)/(B) is from 5:1 to 1:10, and where the amounts stated are based on the sum of all components of the aqueous phase. Preferred polymers (A), hydrophobins (B), amounts and preferred other parameters have already been mentioned above.

The aqueous phases thickened in accordance with the invention generally exhibit marked time-dependent behavior, which means that when the thickened aqueous phase is sheared, its viscosity decreases. After the end of the shear stress, the viscosity of the aqueous phase increases again. When a polymer (A) with thickening action already exhibits time-dependent behavior, the time-dependent effect generally increases as a result of the addition of hydrophobins.

The examples that follow are intended to illustrate the invention in detail:

Thickening Polymers (A) Used

For the experiments, the polymers (A) listed below were used. A1 to A3 are three different commercial alkali-soluble dispersions of acrylates, A4 and A5 are precipitation polymers and A6 is a biopolymer.
Polymer A1: alkali-soluble polyacrylate, associatively thickening aqueous dispersion, pH approx. 3, emulsion polymer
Polymer A2: alkali-soluble polyacrylate, aqueous dispersion, pH approx. 3, emulsion polymer
Polymer A3: alkali-soluble polyacrylate, aqueous dispersion, pH approx. 3, emulsion polymer
Polymer A4: commercial thickener based on lightly crosslinked polyacrylic acid
Polymer A5: commercial thickener based on lightly crosslinked polyacrylic acid
Polymer A6: xanthan

Preparation of the Hydrophobins (B) Used

The hydrophobins used were prepared according to the procedure described in U.S. Patent Publication 2008/0319168. Both a fusion hydrophobin with the complete yaad fusion partner (yaad-Xa-dewA-his; referred to hereinafter as hydrophobin A) and a fusion hydrophobin with a fusion partner truncated to 40 amino acids, yaad40-Xa-dewA-his (hydrophobin B), were used. The hydrophobins were used in the form of an aqueous solution.

Preparation of the Thickened Aqueous Phases

For the examples, an aqueous solution of the hydrophobins (B) was initially charged in each case and then an aqueous solution of the particular polymer (A) was added. The concentrations of (A) and (B) in the aqueous phase used in each case are specified in the tables which follow. If stated in Table 1, the pH of the aqueous phase was subsequently adjusted to the value reported. The details of the experiments are compiled in Table 1.

Measurement of the Viscosity

The viscosity of the aqueous solutions was measured according to the methods DIN 51550, DIN 53018 and DIN 53019 with a customary rotary viscometer (Brookfield® RV-03 viscometer) at a speed of 20 revolutions per minute with spindle no. 64 at 20 ° C. The viscosities were measured immediately after the mixing and after the establishment of the pH. The time-dependent flow behavior was determined, with the viscometer running, by measuring the viscosity as a function of time.

Table 1 shows the initial value in each case.

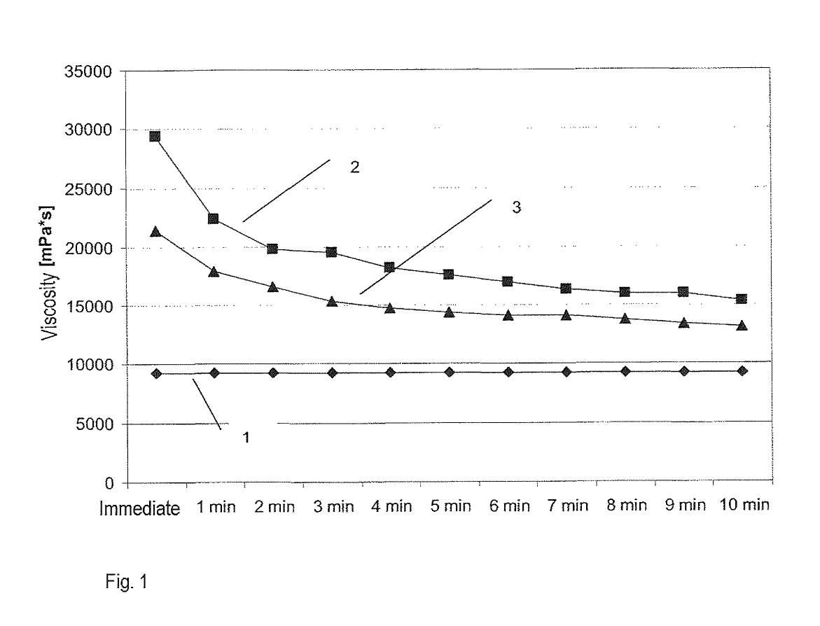

FIG. 1 shows the viscosities of solutions of polymer A1 at pH 9 as a function of time (curve 1: only 1.2% polymer; curve 2: 1% polymer+0.5% hydrophobin A; curve 3: 1% polymer+0.5% hydrophobin B). A clear time dependence of the viscosity of the mixtures of hydrophobin and polymer A1 is discerned, while polymer A1 alone has no time dependence.

All references cited above are incorporated by reference herein in their entirety for all useful purposes.

TABLE 1
Results of the experiments and comparative experiments

| | | | | | | | Visual | |
|---|---|---|---|---|---|---|---|---|
| | Polymer A | | Hydrophobin | | | | assessment | |
| | | Conc. | | Amount | | | of the | |
| | | [% by | | [% by | | Appearance of the | thickening | Initial viscosity³ |
| Example no. | No. | wt.] | Type | wt.] | pH¹ | solution | effect² | [mPa * s] |
| C1 | A1 | 1.2 | — | — | 9 | clear, thick | ++ | 9280 |
| 1 | | 1.0 | A | 0.5 | 9 | clear, thick | +++ | 29440 |
| 2 | | 1.0 | B | 0.5 | 9 | clear, thick | +++ | 21440 |
| C2 | A2 | 1.0 | — | — | 11 | cloudy, thick | ++ | 12160 |
| 3 | | 1.0 | A | 0.5 | 11 | cloudy, thick | ++ | 12800 |
| 4 | | 1.0 | B | 0.5 | 10 | cloudy, thick | ++ | 16000 |
| C3 | A3 | 0.5 | — | — | 11 | clear, thick | + | 3200 |
| 5 | | 0.5 | A | 0.5 | 9 | clear, thick | +++ | 30080 |
| 6 | | 0.5 | B | 0.5 | 10 | clear, thick | +++ | 56960 |
| C4 | A4 | 0.2 | — | — | 10 | slowly clearing, thick | + | 6400 |
| 7 | | 0.2 | A | 0.5 | 10 | slowly clearing, thick | ++ | 24960 |
| 8 | | 0.2 | B | 0.5 | 10 | slowly clearing, thick | ++ | 16640 |
| C5 | A5 | 0.050 | — | — | — | slowly clearing, thick | ++ | 640 |
| 9 | | 0.050 | B | 0.5 | 10 | slowly clearing, thick | ++ | 2240 |
| C6 | A6 | 0.5 | — | — | 11 | cloudy, thick | + | 4160 |
| 11 | | 0.5 | A | 0.5 | 11 | cloudy, thick | + | 5440 |
| 12 | | 0.5 | B | 0.5 | 11 | cloudy, thick | + | 5120 |

¹In the case of polymers A1 to A5, the pH was adjusted to the value with the aid of NaOH.
²Visual assessment of the thickening effect (+ slight thickening, ++ significant thickening, +++ very significant thickening)
³Viscosity immediately after mixing

1. A method for thickening aqueous phases comprising incorporating into the aqueous phase a synergistic mixture comprising
a water-soluble polymer (A) with thickening action and
a hydrophobin (B)
in a weight ratio (A)/(B) of 5:1 to 1:10.

2. The method of claim 1, wherein the (A)/(B) ratio is 3:1 to 1:2.

3. The method of claim 1, wherein the polymer (A) is incorporated in an amount of 0.01% to 2.5% by weight based on the sum of all components of the aqueous phase.

4. The method of claim 1, wherein the hydrophobin (B) is used in an amount of 0.1% to 2.5% by weight based on the sum of all components of the aqueous phase.

5. The method of claim 1, wherein, after the aqueous phase has been thickened, the thickening action is degraded again by adding to the aqueous phase at least one agent which is capable of cleaving peptide bonds in the hydrophobin.

6. The method of claim 5, wherein a protease is used to cleave the hydrophobin.

7. The method of claim 1, wherein the polymer (A) is a polysaccharide.

8. The method of claim 1, wherein the polymer (A) is an alkali-soluble polymer comprising (meth)acrylic acid units and (meth)acrylic ester units.

9. The method of claim 1, wherein the polymer (A) is a hydrophobically associative polymer.

10. A synergistic composition comprising
an aqueous phase,
0.01% to 2.5% by weight of a water-soluble polymer (A) with thickening action, and
0.1% to 2.5% by weight of a hydrophobin (B),
wherein the weight ratio (A)/(B) is 5:1 to 1:10, and where the amount of (A) and (B) is based on the sum of all components of the aqueous phase.

11. The synergistic composition of claim 10, wherein the (A)/(B) ratio is 3:1 to 1:2.

12. The synergistic composition of claim 10, wherein the polymer (A) is a polysaccharide.

13. The synergistic composition of claim 10, wherein the polymer (A) is an alkali-soluble polymer comprising (meth)acrylic acid units and (meth)acrylic ester units.

14. The synergistic composition of claim 10, wherein the polymer (A) is a hydrophobically associative polymer.
